(19) United States
(12) Patent Application Publication
(10) Pub. No.: US 2018/0270548 A1
(43) Pub. Date: Sep. 20, 2018

(54) WATER QUALITY MONITORING SYSTEM AND METHOD

(71) Applicant: Water Clinix of America, Inc., Waukesha, WI (US)

(72) Inventors: Christopher Robert Richter, Milwaukee, WI (US); Robert Gustave Richter, JR., Waukesha, WI (US); Edgar Alejandro Duarte Ortega, Milwaukee, WI (US)

(21) Appl. No.: 15/923,426

(22) Filed: Mar. 16, 2018

Related U.S. Application Data
(60) Provisional application No. 62/472,887, filed on Mar. 17, 2017.

(51) Int. Cl. H04Q 9/00; G01N 27/06; G01N 33/18

(52) U.S. Cl. CPC H04Q 9/00; G01N 27/06; H04Q 2209/40; H04Q 2209/84; G01N 33/1886

(57) ABSTRACT

A sensor system includes a sensing device that has a hardware application program interface (API) that is manipulated by a cloud service to measure certain parameters using a variety of attached sensors. The hardware API creates a communication interface between the sensing device and the cloud service that permits the cloud service to manipulate how the device's hardware components acquire raw data from one or more attached wired or wireless sensors. In certain embodiments data acquisition with the sensing device may be entirely cloud initiated permitting sensing device to be controlled to collect data using any type of sensor without the sensing device requiring any sensor-specific hardware or locally-stored programming specific to a particular type of sensor coupled to the device.

### CROSS-REFERENCE TO RELATED APPLICATION

The present application is a non-provisional of, and claims priority to, U.S. Provisional Patent Application Ser. No. 62/472,887, filed Mar. 17, 2017, the disclosure of which is incorporated herein by reference in its entirety.

### BACKGROUND OF THE INVENTION

The present invention relates generally to a water quality sensor system and method of monitoring water quality, more particularly, to a system and method that permits characteristics of an end-user water supply to be monitored in real time via a phone or tablet. The disclosed system and method also permits a user to monitor other aspects of their water system or any other medium in real-time via one or more different types of sensors coupleable to universal sensor inputs of a monitoring device.

Existing devices used to determine water quality typically include one or more water quality meters or sensors that are inserted into the water supply network of the water supplier (e.g., a municipal water utility or other large-scale water supplier) to measure the water quality within the network. While these sensor systems monitor characteristics of the water quality as it leaves the water supplier, the sensor systems cannot account for contamination that may occur as the water travels between the water supplier and the end user.

An end user who has concerns regarding the quality of the water he or she receives from the water supply network may elect to install an in-line filtering device that is configured to filter the incoming water before it is output from one or more outlets positioned within the home. While these filtering devices improve the water quality from the water supply network, the filtering devices typically do not include integrated water sensors, cannot detect an increase in contaminants in the incoming water supply that may not be completely removed in the filtering device, and do not provide real-time data regarding the water quality characteristics that is accessible to a user remotely located relative to the filtering device.

Commercial and industrial systems such as car washes or food processing centers may include an integrated sensor system that monitors incoming water characteristics to ensure that the water used meets particular water quality thresholds. These sensor systems assess water quality by acquiring an impedance or resistance reading from the water using a pair of electrodes. A voltage differential is created across the electrodes, which causes an electric current to flow in the water from one electrode to the other electrode. The current is generated at a particular, static frequency that is known to be correlated with a desired water quality characteristic and is preprogrammed into the sensor processor or controller. Water quality measurements are acquired by manually pushing a button on the sensor system, which causes the device to generate the voltage potential between the sensor electrodes. Due to their high cost, these types of sensor systems are cost prohibitive for residential applications.

Another factor that contributes to the relative high cost of water sensor systems is the incremental cost associated with analyzing different types of water characteristics. Prior art systems utilize different types of sensors for measuring different water characteristics, with each sensor associated with a dedicated processor specifically programmed to output a particular, static frequency signal to the sensor known to be tied to a particular water quality characteristic. For example, a sensor processor configured to detect one type of contaminant in water may be programmed to subject a sample to one static frequency, while a sensor processor configured to detect a different type of contaminant may be programmed to subject the sample to a different static frequency. The greater the desired detection functionality of the sensing device, the higher the total cost of the system becomes as the inclusion of each sensor and associated processor incrementally increases the total sensor cost. Adding more sensors to the system also increases the overall size of the device, as the device must be configured with a dedicated input port corresponding to each sensor.

It would therefore be desirable to have a low-cost system and method capable of analyzing and monitoring characteristics of a given medium in real time at a remote location relative to the monitored medium.

### BRIEF DESCRIPTION OF THE INVENTION

In accordance with one aspect of the invention, a sensing device includes at least one input port, a data acquisition system (DAS) in communication with the at least one input port, and a main connectivity module coupled to the DAS. The main connectivity module is configured to receive a measurement command from a source external to the sensing device. The measurement command includes at least one DAS setting specific to a type of sensor coupled to the at least one input port. The main connectivity module is also configured to execute the at least one DAS setting by causing the DAS to stimulate a sensor coupled to the at least one input port, receive raw data from the stimulated sensor, and transmit the raw data to a processor external to the sensing device.

In accordance with another aspect of the invention, a remotely-controlled sensor system includes a remote database and a sensing device having a main connectivity module, a data acquisition system (DAS) in communication with the main connectivity module, and a first input port in communication with the DAS. The first sensor is coupled to the input port. The system also includes a data processing system in communication with the sensing device and the remote database. The data processing system includes a processor configured or programmed to identify a sensor type of the sensor using data stored on the remote database and generate a measurement command based on the identified sensor type, where the measurement command includes at least one DAS setting that defines a hardware configuration for the sensing device. The processor is also configured or programmed to transmit the measurement command to the sensing device and receive raw data from the sensing device, where the raw data includes data acquired by the stimulating the sensor in accordance with the at least one DAS setting.

In accordance with another aspect of the invention, a method of acquiring data from a sensor includes identifying a sensor type of a sensor coupled to a sensing device using data stored within a database and generating a measurement command within a processor external to the sensing device, the measurement command including at least one data acquisition system (DAS) setting configured to stimulate the sensor based on the identified sensor type. The method also includes transmitting the measurement command from the external processor to a microcontroller on the sensing device and executing the measurement command within the microcontroller by configuring at least one DAS component on the sensing device to stimulate the sensor in accordance with the at least one DAS setting. The method further includes receiving raw data from the stimulated sensor at the sensing device and transmitting the raw data from the sensing device to the external processor in real time.

These and other advantages and features will be more readily understood from the following detailed description of preferred embodiments of the invention that is provided in connection with the accompanying drawings.

### BRIEF DESCRIPTION OF THE DRAWINGS

The drawings illustrate preferred embodiments presently contemplated for carrying out the invention.

In the drawings:

FIG. 1 is a schematic block diagram of a cloud-connected sensing system, according to one embodiment of the invention.

FIG. 2 is an exemplary graphical user interface usable in connection with the sensing system of FIG. 1, according to one embodiment of the invention.

FIG. 3 is an exemplary graphical display of a water quality index, according to one embodiment of the invention.

FIG. 4 is an exemplary administrator interface usable in connection with the sensing system of FIG. 1, according to one embodiment of the invention.

FIG. 5 is a front view of a sensing device usable in the sensing system of FIG. 1, according to one embodiment of the invention.

FIG. 6 is a bottom view of a sensing device usable in the sensing system of FIG. 1, according to one embodiment of the invention.

FIG. 7 is a side view of a sensing device usable in the sensing system of FIG. 1, according to one embodiment of the invention.

FIG. 8 is a schematic block diagram of a wireless or remote sensor usable in the sensing system of FIG. 1, according to another embodiment of the invention.

FIG. 9 is a technique for analyzing and monitoring characteristics of a medium utilizing the sensing system of FIG. 1, according to one embodiment of the invention.

FIG. 10 is a schematic diagram of an exemplary implementation of a water sensing system, according to one embodiment of the invention.

FIG. 11 is a schematic diagram of an exemplary implementation of a water sensing system, according to another embodiment of the invention.

FIG. 12 is a schematic three-dimensional representation of the water sensing system utilized in FIG. 10, according to one embodiment of the invention.

FIG. 13 is a schematic diagram of a water monitoring network, according to an embodiment of the invention.

### DETAILED DESCRIPTION

Embodiments of the disclosed system differ from typical prior art sensors that include all of the application specific sensors and associated data processing hardware directly on the sensing device. Unlike these prior art systems, the disclosed sensing device includes a hardware application program interface (API) that is controlled by a source external to the sensing device. This external processing source is described herein as a smart cloud management system or cloud-based processing service. However, it is contemplated that the external processing source may a computer, server, or other tangible device that includes processing capabilities similar to the cloud-based processing service described herein. The core software provided on the sensing device is a communication interface to the cloud service that permits the cloud service to manipulate the manner in which the device's hardware components acquire raw data from one or more attached wired or wireless sensors. Thus, the sensing device disclosed herein may be characterized as a cloud-connected hardware device whose API is manipulated by the cloud service to measure certain parameters using a variety of attached sensors.

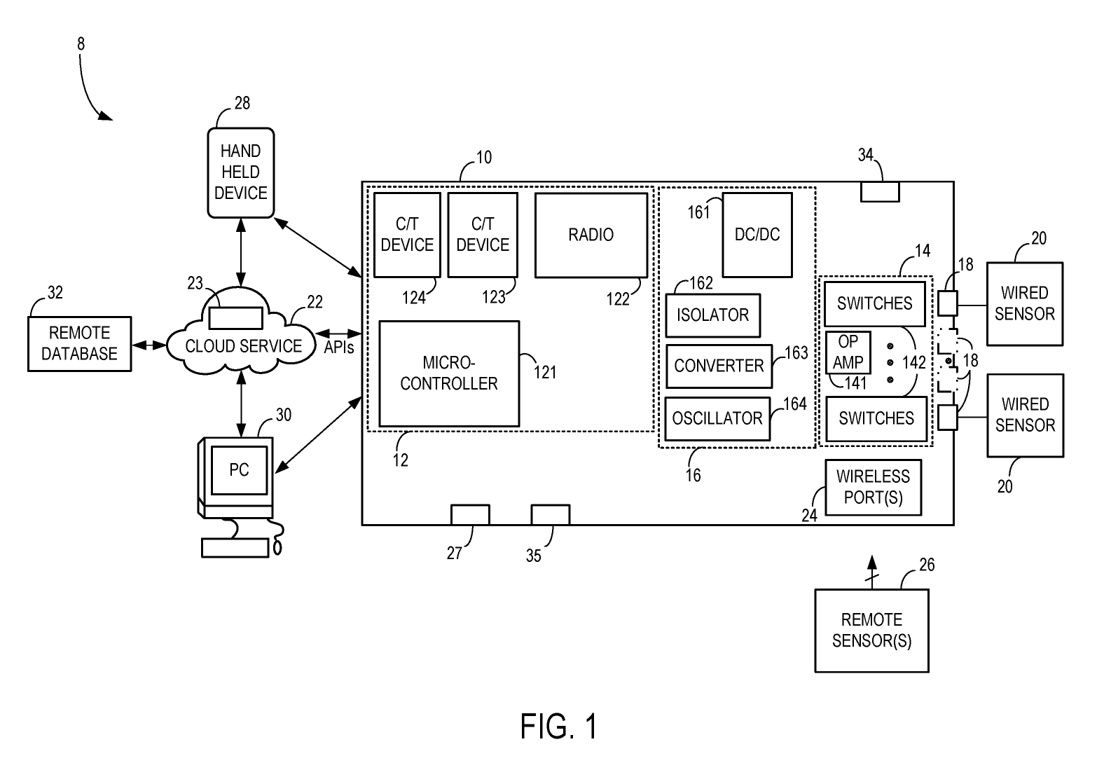

Referring first to FIG. 1, a remotely controlled, cloud-connected sensor system 8 includes a sensing device or sensor device 10 includes hardware components that form a main connectivity subsystem (MCS) or main connectivity module 12 and a multiplexing subsystem (MPS) or multiplexing module 14 coupled to a data acquisition system (DAS) 16, which is a hardware chip that collects data from the physical world. In one non-limiting embodiment, the MCS 12 includes a controller, processor, or microcontroller 121, a radio transmitter/receiver 122, and a plurality of associated frequency control and timing (C/T) devices 123, 124. Radio transmitter/receiver 122 preferably provides a wireless interface, is compatible with a WLAN, BT, and ZB protocols, and includes at least one antenna. While radio 122 and microcontroller 121 are described herein as separate, discrete components, it is contemplated that the functionality of the two devices could be incorporated into a single device structure in an alternative embodiment.

The microcontroller 121 of the main connectivity subsystem 12 is programmed with core software, referred hereafter as the hardware API of the device 10. The hardware API is updatable over the air (OTA) and creates a communication interface between the device 10 and a cloud-based processing service 22. This configuration permits the cloud-based processing service 22 to ping into the sensing device 10 and control the DAS 16 to acquire data in a certain manner based on the particular type of wired sensor(s) 20 and/or wireless sensor(s) 26 attached to the sensing device 10 at any given time. As described in more detail below, the cloud-based processing service 22 broadcasts a measurement command to the microcontroller 121. Upon receipt of the measurement command, the microcontroller 121 executes one or more DAS settings that define a particular hardware configuration of the DAS 16 and the multiplexing system 14 for sensor data acquisition. As a result of the communication protocol between the cloud-based service 22 and the microcontroller 121, every physical port 18 of the device 10 is controllable dynamically via OTA methods. As a result of this configuration, the physical ports 18 are not tied to a specific type of sensor input but rather are universal inputs to which any number of different types of sensors can be connected and appropriately controlled through configuration settings received via the cloud-based processing service 22. Because data acquisition with sensing device 10 is entirely cloud initiated, device 10 is a “sensor agnostic device” meaning that sensing device 10 can be controlled to collect data using any type of wired or wireless sensor without the device 10 having any sensor-specific hardware or locally-stored programming specific to a particular type of sensor coupled to the device 10 as long the cloud-based processing service 22 has specifications for that particular type of sensor.

The microcontroller 121 also includes a static operating system (OS), image, or firmware that includes a mapping of the circuits within the device 10 and identifying information for the wired and wireless ports 18, 24, powers the device 10, and includes a configuration file that stores device-specific identifying information that may include wi-fi credentials, Bluetooth registries, API tokens used to authenticate the device 10, and other similar information.

The microcontroller 121 of MCS 12 includes a central processing unit (CPU), Nonvolatile memory (NVM), and Random-Access Memory (RAM). These components allow the MCS 12 to run the hardware API, which provides a map of what to execute within the DAS 16 and the multiplexing system 14 when the cloud-based processing service or system 22 messages the device 10 with a data acquisition instruction or measurement command. In preferred embodiments, microprocessor 121 does not have sufficient memory to enable substantive latent data storage locally on the device 10 and device 10 does not include any additional memory components. In some embodiments, microcontroller 121 has 10 MB or less of Flash memory. The preferred implementation of microprocessor 121 has 1 MB of Flash memory and may be provided as model number STM32F407VGT6 microcontroller manufactured by ST Microelectronics. However, it is contemplated that microcontroller 121 may be any suitable controller or processor component that meets design specifications. The MCS 12 connects to the DAS 16 via a serial bus. In one embodiment, the device 10 includes an I2C (Inter-Integrated circuit), however, embodiments are not limited to such serial bus. Alternative embodiments may include SPI (Serial peripheral interface) or other known serial communication implementations.

In one non-limiting embodiment, the DAS 16 includes a DC/DC converter 161, a digital isolator 162, an impedance converter/network analyzer 163, and a clock oscillator 164. According to one embodiment, the impedance converter 163 of DAS 16 is comprised of several analog submodules. These submodules include a local DSP (Digital signal processing) logic to compute a DFT (Discrete Fourier Transform), a DAC, and an ADC, which together form a Direct Digital Synthesizer (DDS). In one non-limiting embodiment, impedance converter 163 is a 250 kSPS, 12-Bit Impedance Converter, Network Analyzer such as model number AD5934 manufactured by Analog Devices. However, an alternative ADC may be used. Alternative embodiments of the invention might utilize an alternative integrated circuit that allows DDS techniques and other analog to digital conversion techniques as desired.

The multiplexing subsystem 14 routes the output of the DAS 16 to one or more physical ports 18 on the device 10. The MPS 14 is constructed from a switching matrix of low “On” resistance and low “Off” current leakage analog switches 142. Switches 142 may be CMOS, octal single-pole, single-through switches or other known switching structures. The multiplexing subsystem 14 may also include a CMOS Op Amp 141, in one exemplary embodiment. Components of the MPS 14, DAS 16, and the MCS 12 implement a version of an I2C serial interface or bus that allows the components to communicate and allows the MPS 14 to set up the appropriate sensor and DAS settings. The MCS 12 can then command via the serial bus which physical ports 18 to route to the DAS 16. The switching matrix routes both the stimulation signal coming from the DAC and the signal coming from the stimulated sample to the impedance converter 163 of the DAS 16.

According to alternative embodiments, sensing device 10 may include a single input port 18 or any n-number of multiple input ports 18. Each port 18 is configured for connection to a wired sensor 20. One or more of the ports 18 may be configured as a universal input port to permit connection by multiple different types of sensors. In one embodiment, the universal input port is a four-pin inline connector. In alternative embodiments, sensing device 10 may include different types of ports. For example, one or more ports 18 may be configured as a four-pin inline connector, a two-pin inline connector, a BNC connector, or any other known connector configuration. In yet other embodiments, sensing device 10 may be provided with a coupling device (not shown) that is received within one of the ports 18 and that includes input connectors for several different types of sensor configurations. It is to be understood that sensing device 10 includes numerous additional components, such as passive components, that have been omitted from FIG. 1 for clarity purposes.

The MCS 12 is configured to send a signal to the DAS 16 to generate the stimulation signal in accordance with the measurement command received from the cloud-based processing service 22. The stimulation signal may be provided any frequency allowed by the hardware of device 10. The output of the DAS 16 signal gets buffered and filtered to stimulate the external sample routed by the MPS 14. The resulting signal from the stimulation is read by the impedance converter 163 at the DAS 16. In some cases, the DAS 16 performs a DFT algorithm in order to obtain the complex impedance of the stimulated sample. This raw data is transmitted through the MCS 12 to the cloud-based processing service 22, where it is processed to obtain an estimate of the physical properties in the measured medium, such as, for example, electrical conductivity or other present contaminants. The cloud-based processing service 22 is not limited to using complex impedance as its source for parameter estimation and it is contemplated that alternative implementations may use other types of signals such as light or sound signals for parameter estimation.

According to various embodiments, wired sensors 20 may include any type of water sensor configured to measure a particular physical feature, parameter, or characteristic of the medium. Where the medium is water, such sensors 20 may include, as non-limiting embodiments, conductivity probes, capacitance sensors, ultrasonic sensors, optical sensors, and the like. Exemplary water quality parameters include temperature, pH, oxidation-reduction potential (ORP), conductivity or salinity, and dissolved oxygen (DO). Water quality parameters may also include detection of total dissolved solids (TDS) including bicarbonates, chlorides, sulfates, Ammonia, Bromide, Calcium, Cupric, Fluoride, Fluoroborate, Iodide, Lithium, Magnesium, Nitrate, Perchlorate, Potassium, Silver, and Sodium, as a non-limiting list. Such sensors may also be configured to monitor for other types of contaminants in the water, including lead, arsenic, and other health hazards such as prescription or over-the-counter drugs, herbicides, pesticides, other chemicals, and detergents, as a non-limiting list. Additional types of sensors may be used, such as, for example, sensors that include membranes for detecting pathogens and biological components that might be hazardous for humans. Exemplary pathogens include bacteria, viruses, parasitic cysts, e. coli, cryptosporidium, Legionella, and giardia. Sensors may also be configured to detect other contaminants including volatile organic compounds (VOCs), total trihalomethanes (TTHMs), and/or Methyl tert-butyl ether (MTBEs).

While sensing device 10 is described herein with respect to sensors configured to measure water characteristics, it is contemplated that the techniques described herein may be extended for use in measuring characteristics of other types of liquids as well as to sensors capable of measuring parameters of gases or other substances. As just a few alternative examples, sensing device 10 may be used in connection with one or more attached sensors to monitor air quality in real time or detect the presence of a particular gas or contaminant in an ambient airflow (e.g., radon, carbon monoxide, and the like). Sensing device 10 may also be used for air-based monitoring in the context of health and wellness applications and monitor for pollen, dust, or other types of allergens. Sensing device 10 may also be configured to measure physical characteristics of a medium including flow rate, total gallons, water pressure, and the like.

In addition to physical ports 18, sensing device 10 can also include any number of wireless or virtual ports 24 that permit wireless or remote sensors 26 to be connected to the network management layer of the main connectivity subsystem 12. Remote sensors 26 may include a water detection device configured to monitor for a water leak, a temperature sensor, a pressure sensor configured to monitor the current salt level in the brine tank of a water softener system, and a flow meter or flow totalizer, as non-limiting examples. Data received from remote sensors 26 on virtual ports 24 may be communicated to sensing device 10 through any known wireless protocol, including, for example, a cellular or long-range protocol, Bluetooth low energy (BLE), and LoRa as non-limiting examples. Data received through virtual ports 24 is transmitted by the main connectivity subsystem 12 to cloud-based processing service 22, handheld device 28, and/or computer 30 using any of these wireless protocols. Alternatively, handheld device 28 and/or computer 30 may be configured to receive/transmit data to and from cloud-based processing service 22 and remote network 32 via a hard-wired internet connection.

The cloud-based processing service 22 includes at least one processor 23 programmed with control software or scripts that include measurement commands that manipulate the internal hardware configuration of device 10 to acquire raw data via wired sensor(s) 20 and/or remote sensor(s) 26. These measurement commands include sensor-type specific DAS settings that dictate how the internal hardware components of device 10 are operated to perform measurements or acquire raw data using a particular wired or wireless sensor 20, 26 coupled to the device 10. The DAS settings are communicated to the device 10 by way of a real-time, persistent connection (e.g., an MQTT tunnel) between the cloud service 22 and the device 10. In the embodiment described herein, the DAS setting information is not stored locally on the device 10. Instead, device 10 receives the setting information from the cloud service 22 in real time and executes the setting information in real time through the hardware API file residing on the microprocessor 121. The setting information is cycled through the internal hardware components of device 10 and the sensor 20, 26 is stimulated to acquire data. In an alternative embodiment, sensing device 10 may include the storage capability to save a particular sensor configuration or DAS settings corresponding to a particular measurement command received from the cloud-based service 22, which will permit the device 10 to acquire data from attached sensor(s) 20, 26 while offline or disconnected from the cloud-based processing service 22.

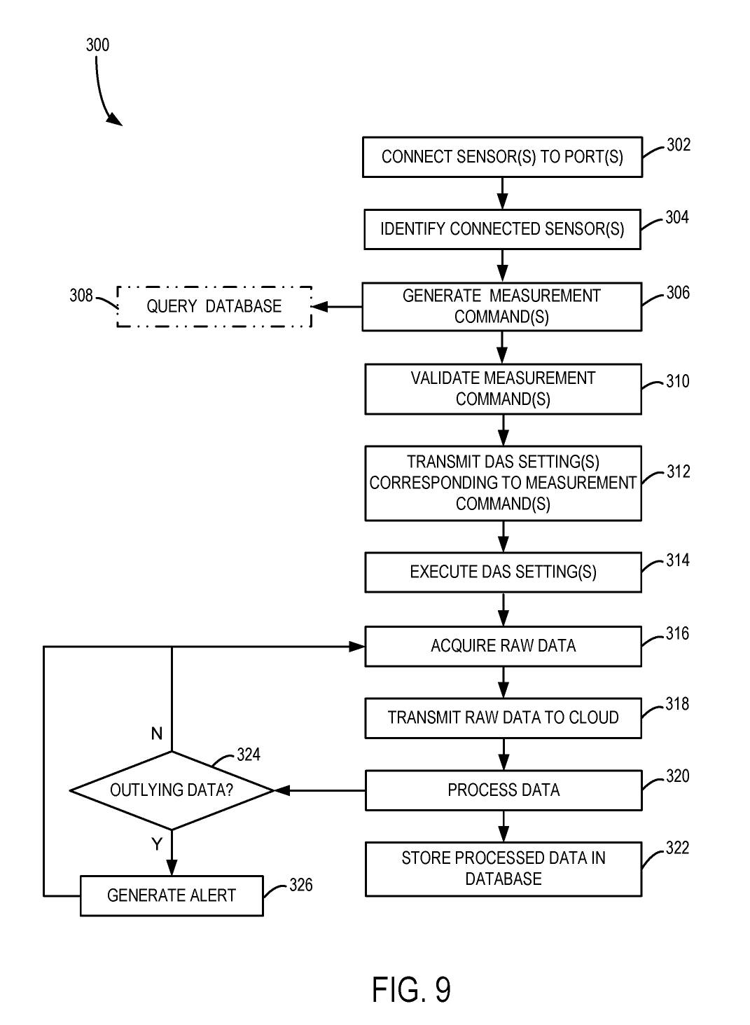

Data measurements occur in real time with a persistent connection between the device 10 and the cloud-based processing system 22. As described in further detail with respect to FIG. 9, after the raw data is acquired, the data is transmitted to the cloud service 22 for substantive processing. The acquired raw data is not stored locally on device 10 and resides on the device 10 only temporarily. The raw data is transmitted in real time to the cloud service 22 where it is processed by the cloud service 22 and stored to the remote database 32 for subsequent delivery to the handheld device 28 or PC 30.

The control software or scripts that reside on the cloud-based processing service 22 include a number of topics that define certain actions for the device 10. The script associated with each of these topics interacts with the hardware API of device 10 by way of a measurement command that dictates certain DAS settings for the device 10 including, for example, defining the port 18 on device 10 through which data is to be acquired, setting internal gain of the impedance converter 163, setting system gain or external gain (e.g., by changing the matrix switch and selecting a different feedback resistor for the OPAMP circuit), setting analog channel, defining frequency settings such as a start frequency, an end frequency, and/or frequency intervals, setting voltage, defining the total number of samples, or other measurement variables. The cloud-based processing service 22 broadcasts or publishes a particular topic and the hardware API of the device 10 subscribes to the broadcast/published topic. The microprocessor 121 of device 10 receives DAS setting information from the cloud-based processing service 22 on this subscription basis.

The number and type of topics included on the cloud-based processing service 22 may be added to or changed over time based on any number of factors including desired functionality of device 10, the types of available wired and wireless sensors 20, 26, or desired data measurements, as non-limiting examples. As one example, cloud-based processing service 22 may include a sweep topic that configures device 10 to sweep a frequency spectrum (e.g., for impedance spectroscopy). Cloud-based processing service 22 may also include a system calibration topic that is broadcast when a device 10 initially comes online. This system calibration topic may populate an initial database for the given device 10 and transmit a measurement command that initiates a series of readings from one or more components on device 10 (e.g., a 1 k resistor and a 10 k resistor). The acquired calibration data would be transmitted back to the cloud-based processing service 22 and stored within remote database 32. The stored data would later be accessed by the cloud-based processing service 22 and used to adjust later acquired data readings from the device 10. The system calibration topic thus defines a device-specific calibration procedure that generates calibration data for the overall device 10 rather than a particular sensor that might later be coupled to that device 10.

Another possible topic is a conductivity calibration topic that causes device 10 to implement a sensor-specific calibration routine (e.g., a traditional, in-field calibration against a known solution) wherein data acquired by device 10 using that particular sensor is transmitted to the cloud-based processing service 22 and processed to define calibration data specific to the connected sensor. Cloud-based processing service 22 may also include a conductivity reading topic that generates a measurement command to acquire data at a specific frequency.

In addition to topics that include measurement commands, the cloud-based processing service 22 may include topics that dictate certain operational aspects of device 10. For example, the cloud-based processing service 22 may broadcast a sleep topic that directs microprocessor 121 to control internal components of device 10 enter into a deep sleep mode. As yet another examples, the cloud script may include an initial device initialization or sign on topic, a wi-fi change request that tells the device 10 to change wi-fi networks in the event of low connectivity, a status indicator topic used by the cloud service 22 to determine that a process is currently running on the device 10, a topic directed to measuring up-time or network health, a battery status topic that gathers the battery voltage from the device 10 and permits remote monitoring of battery health via the cloud-based processing service 22.

When the cloud-based processing service 22 broadcasts or publishes a particular topic to a device 10, the broadcast includes a topic-specific ID and a packet of DAS settings associated with that particular topic. Device 10 reads the topic-specific ID and accesses instructions stored within the hardware API on microprocessor 121 for the particular topic-specific ID. These instructions dictate how microprocessor 121 is to apply the DAS settings received from the cloud-based processing service 22. As just one example, cloud-based processing service 22 broadcasts a sweep topic that includes a topic-specific ID “SWEEP_TOPIC.” Upon receipt of the broadcast, microprocessor 121 accesses its hardware API for saved instructions specific to “SWEEP_TOPIC.” These saved instructions tell the microprocessor 121 how to interpret and apply the DAS settings broadcast from the cloud-based processing service 22. For example, the saved instructions might instruct the microprocessor 121 to use a DAS setting labeled ‘gain’ to set the gain for a particular port 18 at a value transmitted with that ‘gain’ DAS setting.

Cloud-based processing service 22 broadcasts or publishes a particular topic to a device 10 using a promise and resolve programming strategy. When the topic is broadcast, a unique ID or key is created for the broadcast and stored by the cloud-based processing service 22. The raw data transmitted by the device 10 to the cloud-based processing service 22 in response to the broadcast is linked to the unique ID or key tied to the topic broadcast.

Once connected to the cloud-based processing service 22, main connectivity subsystem 12 configures the DAS 16 and multiplexing subsystem 14 to acquire data, thereby permitting the sensing device 10 to be configured to acquire data using the physically and/or wirelessly attached sensor(s) 20, 26. In this manner, the sensing device 10 is effectively controlled via the cloud-based processing service 22 in a manner that permits the cloud-based processing service 22 to define how data is acquired from any of the physical ports 18 and any of the virtual ports 24 of the sensing device 10 regardless of the type of sensing device that is connected to a specific port 18, 24. Accordingly, any number of different types of sensors 20, 26 can be interchangeably coupled to ports 18, 24.

According to various embodiments, a user can select cloud-based processing service 22 and/or remote network 32 to perform the various functions, analytics, and services described herein. For example, one can decide to have a provider “X” for data storage and a provider “Y” for the analytics and “Artificial Intelligence” service to analyze the data. Alternatively, data storage and analytical services may be provided by a common cloud-based processing service 22 such as, for example, IBM's Cloudant service and IBM's Watson service. Any of the two services could be sources from any other provider. For example, Google's Firebase may be utilized for the Cloudant alternative and Watson could be used for the analytics layer.

Sensing device 10 is configured to operate based on a particular configuration of wired sensors 20 and/or remote sensors 26 that is defined when a user accesses an application or user interface on a handheld device 28, such as a cellular phone or tablet, or a computer 30. In order to permit cloud-based processing service 22 to configure sensing device 10 to acquire data using the current configuration of wired sensors 20 and/or remote sensors 26, the user access a device configuration protocol using a graphical user interface (GUI) through which the user is able to identify the type of sensor coupled to each port 18, 24. It is contemplated that the GUI may include a drop-down menu that lists the various sensor types and/or a schematic diagram illustrating the various available physical ports 18 and virtual ports 24 on the sensing device 10 to facilitate this correlation process.

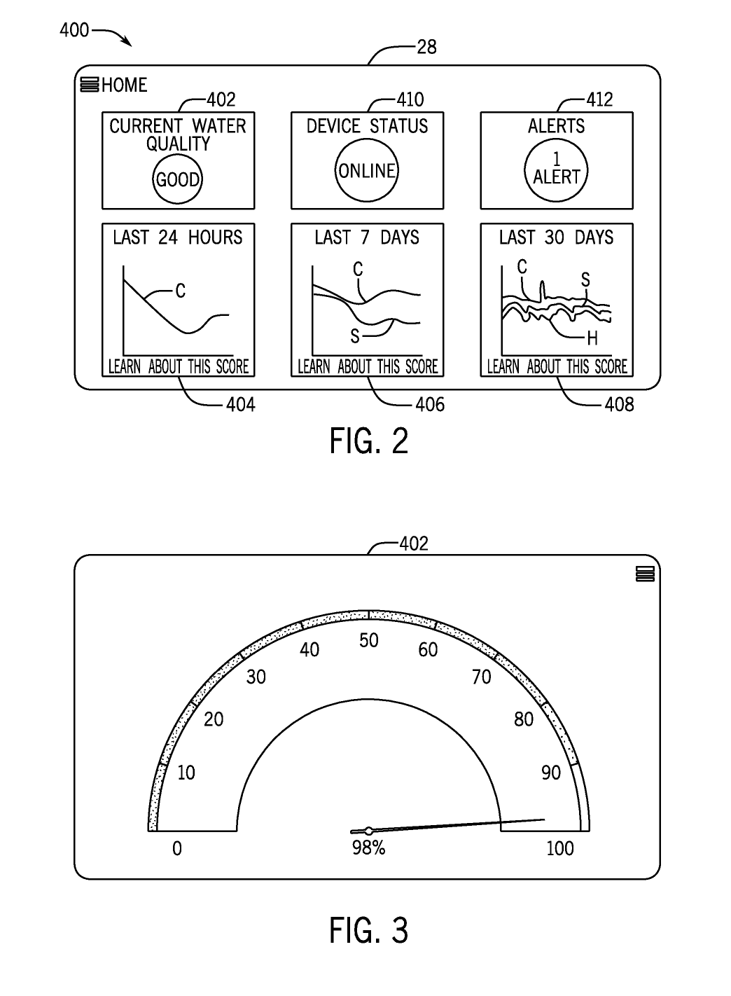

Referring now to FIG. 2, a graphical user interface (GUI) 400 is illustrated according to an embodiment of the invention. GUI 400 is designed to be displayed on the handheld device 28 or computer 30 of FIG. 1. The GUI 400 includes a plurality of sections designed to receive and communicate a variety of information to and from an operator. These sections may include one or more display sections 402, 404, 406, 408, 410, and 412 configured to display alerts, data reports, device status, and other information to the user. Data may be displayed in one or more of display sections 402, 404, 406, 408, 410, and 412 in real time and relative to historical charts, as a non-limiting example.

In the illustrated example, display section 402 conveys the real time or current water quality parameter or index to the user textually (e.g., “GOOD”, “POOR”) and optionally in combination with a color scale (e.g., “GOOD” displayed green, “POOR” displayed red). As used herein, the term “index” refers to a scale that rates water quality within historical norms. GUI 400 may display water quality parameter or index in alternative ways within display section 402, such as in terms of the overall effectiveness of a drinking water system, as shown in FIG. 3. One or more additional display sections 404, 406, 408 may be configured to graphically depict changes in one or more water quality parameters over time. Display section 404, for example, illustrates changes in water quality parameter C over the last twenty-four hours. Display section 406 illustrates exemplary changes in water quality parameters C and S over the last seven days. The graph depicted in display section 408 illustrates exemplary changes in water quality parameters C, S, and H over the past 30 days.

The GUI 400 also includes a menu 414 that permits a user to access a settings section wherein the user can define a user profile that includes identifying information for the user such as basic contact information for the user, a username and password, geographic location, and other account information. The settings section may also include input fields where the user can define share settings for the device 10 such as sharing or database permissions that extend sharing permissions from one authenticated user (i.e., the owner of the device 10) to any n number of other individuals. The sharing permissions may define permissions that allow other individuals to view acquired data and define configurations for the device 10, as examples. Through a selection available in the settings section GUI 400, a user may choose to keep his or her water data private or anonymously share the data with a remote database or network 32, allowing others to access the data over the internet. The user profile data and share permissions may be stored on the remote database 32 and/or by the cloud-based processing service 22.

The menu 414 also permits a user access to a device selector section that allows a user to select from a listing of sensing devices, such as device 10, which the user has configuration permissions for. After the user has selected a particular device 10, the GUI 400 will display a device configuration section wherein the user is permitted to identify any wired sensor(s) 20 coupled to the physical ports 18 of device 10 and also identify any wireless sensor(s) 26 to be associated with the virtual ports 24 of device 10. With respect to the wired sensor(s) 20, the device configuration section may include a series of drop down menus that allow the user to select or change a sensor 20 coupled to each port 18. With respect to the virtual ports 24, the device configuration section may include a drop-down menu corresponding to each of then number of virtual ports and permit the user to select or change a sensor coupled to each port. The device configuration section may also include one or more drop down menus that allow the user to identify the type of medium that a given sensor 20, 26 is sensing (e.g., untreated water, softened water, water output from a reverse osmosis system, etc.). The drop-down menus provided within the device configuration section may list available sensors by sensor type, make, and/or model number. GUI 400 accesses the listing of available sensors from data stored on the remote database 32 and/or cloud-based processing service 22, according to alternative embodiments.

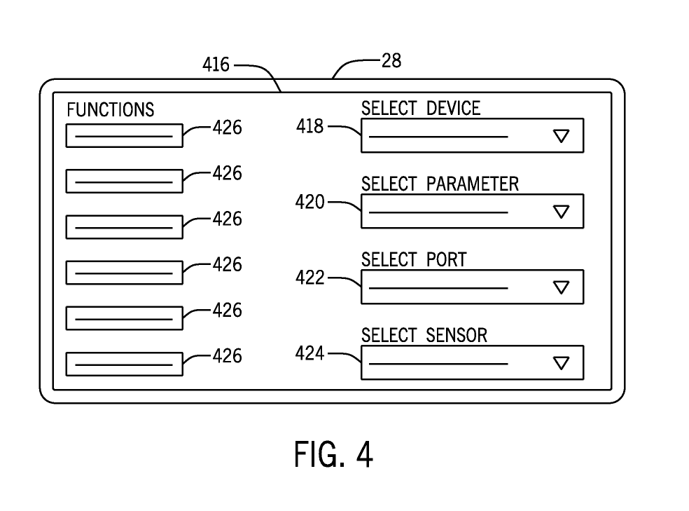

In some embodiments GUI 400 includes an administrator interface similar to administrator interface 416 of FIG. 4, which permits a user to control data acquisition from multiple sensing devices from the user's handheld device 28 or computer 30. In alternative embodiments, the administrator interface 416 is provided on a GUI that is separate and apart from GUI 400. The administrator interface 416 includes a drop down menu 418 that includes a listing of sensing devices, a drop down menu 420 that includes a listing of measurement parameters, a drop down menu 422 that enables a user to select a port (wired or virtual) on the selected sensing device, and another drop down menu 424 that includes a listing of different types of sensors that the user can select from to identify the type of sensor that is coupled to the selected port on the selected sensing device. The administrator interface 416 also includes one or more function selection buttons 426 that correspond to the different topics defined on the cloud-based processing service 22. Pressing or selecting one of these buttons 426 publishes the corresponding topic and transmits the appropriate measurement command to the selected sensing device 10.

Referring again to FIG. 1, sensing device 10 may be battery powered via a rechargeable internal battery (not-shown) or powered through an AC connection port 34 and/or a USB connection port 35. In some embodiments, sensing device 10 acquires data via connected sensor(s) 20, 26 according to a measurement schedule that is determined based on whether the device 10 is operating on battery power or wall power. If operating on wall power, for example, the device 10 is controlled in a continuous or near continuous measurement mode. If operating on battery power, device 10 acquires data from sensor(s) 20, 26 less frequently and may be controlled to enter into a sleep mode between subsequent data acquisition periods. Additionally, or alternatively, control of device 10 while operating on battery power may be limited to smaller measurements such as, for example, conductivity readings.

In some embodiments, sensing device 10 also includes a user interface button 27 that actively interacts with the OS or file system inside device 10. Device 10 may be configured such that a long depression of user interface button 27 erases the file system and one or more short depressions initiate other functions such as waking up the device 10 from a sleep cycle.

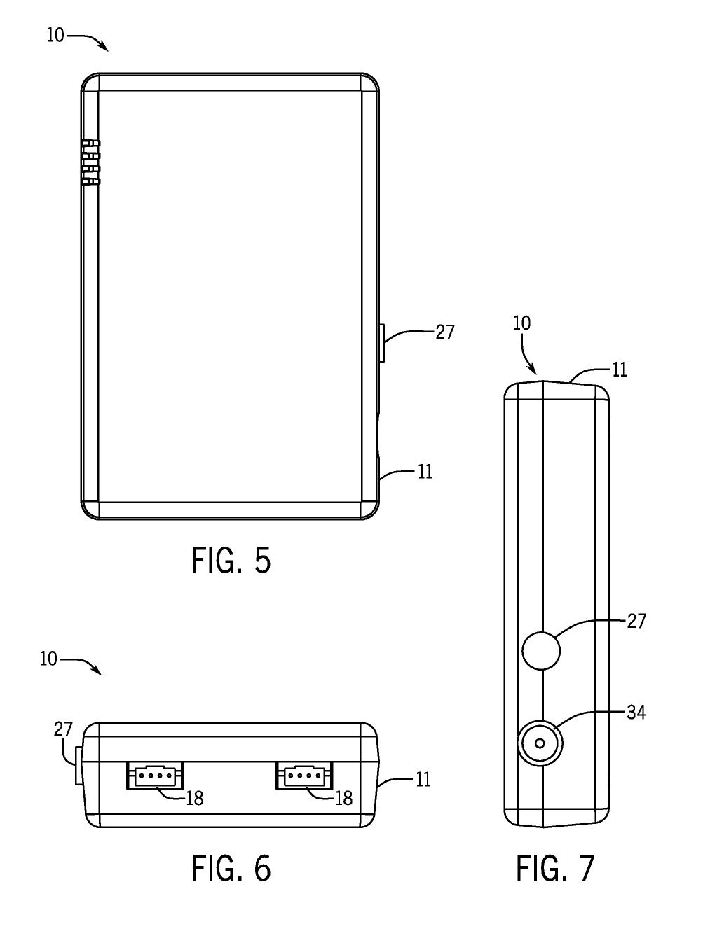

Additional views of the exterior construction of sensing device 10 are provided in FIGS. 5, 6, and 7, according to one embodiment of the invention. Sensing device 10 includes an exterior housing 11 with openings that permit access to the physical ports 18 and AC connection port. User interface button 27 extends through another opening in the housing 11. Sensing device 10 may also include an integrated display (not shown) provided on an external surface of housing 11 and configured to display operational characteristics such as a wireless connection status of the device, battery level indicator, sensor connection status, and any number of water parameters including temperature and water quality, for example.

When sensing device 10 is turned on for the first time, device 10 does not have wi-fi credentials and therefore cannot communicate with the cloud-based processing service 22. Sensing device 10 thus initial operates in a blue tooth mode and a user provides wi-fi credentials to device 10 through GUI 400 (FIG. 2). Thereafter, device 10 transitions over to wi-fi communications for subsequent connection to the cloud-based processing service 22. After device 10 has established wi-fi credentials, device 10 transmits its API token wirelessly to the cloud-based processing service 22 once device 10 comes online. In some embodiments, the API token is transmitted via a MQTT broker.

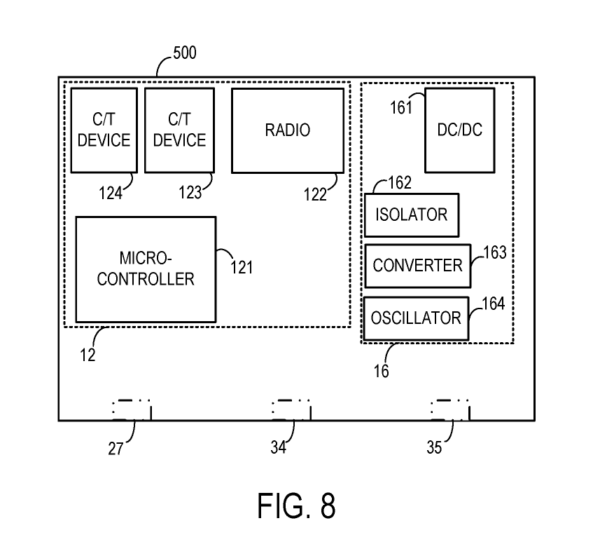

Referring now to FIG. 8, a wireless or remote sensor 500 is illustrated in accordance with an embodiment of the invention. Remote sensor 500 is usable with the sensing device 10 of FIG. 1 and may be used in place of or in addition to remote sensor 26. Remote sensor 500 includes a MCS 12 and a DAS 16 that are configured in a similar manner and include similar components to sensing device 10, with the exception that the hardware API of MCS 12 in remote sensor 500 would not include mapping for a multiplexing system. Common part numbering from FIGS. 1 and 8 is used as appropriate. By integrating MCS 12 and DAS 16 directly within wireless sensor 500, the wireless sensor 500 can be operated as a standalone device that is controlled directly from the cloud-based processing service 22. Remote sensor 500 is provided with a device-specific ID code or API token that is stored on the local API hardware or file system of the microcontroller 121. Cloud-based service 22 can then transmit measurement commands including DAS settings to remote sensor 500, which are executed within the remote sensor 500 in a similar manner as described with respect to sensing device 10.

Referring now to FIG. 9, a technique 300 for monitoring water quality characteristics is illustrated, according to one embodiment of the invention. At step 302 one or more wired sensors 20 is connected to one or more physical ports 18 of sensing device 10. At step 304, a user accesses the user interface/application (e.g., GUI 400 of FIG. 2) and identifies the type(s) of wired sensor(s) 20 connected to the physical ports 18. Through GUI 400 (FIG. 2), the user may also identify any remote sensors 26 associated with sensing device 10 during this step. As one non-limiting example of the above-described sensor configuration protocol, the user might select physical port 1 of the sensing device 10 and associate the port with a conductivity sensor and select physical port 2 of the sensing device 10 and associate the port with a pH sensor. The sensor configuration information is transmitted from the handheld device 28 or computer 30 to the cloud-based processing service 22.

Based on the identified sensor configuration, the cloud-based processing service 22 generates one or more measurement commands in accordance with the sensor configuration at step 306, which will in turn transmit to MCS 12 the appropriate DAS settings through which the DAS 16 and multiplexing subsystem 14 are controlled to properly acquire raw data using the sensor(s). Following from the earlier example, the cloud-based processing service 22 may generate a first measurement command that includes one or more DAS settings specific to the identified make/model of conductivity sensor coupled to physical port 1 and a second measurement command that includes one or more DAS settings specific to the identified make/model of pH sensor coupled to physical port 2.

The wired sensor(s) 20 and/or remote sensor(s) 26 are used by sensing device 10 to collect raw data during step 318 that is used by cloud-based processing service 22 to calculate and monitor characteristics of the monitored medium. Where the medium is water, the monitored water characteristics may include conductivity, oxidation-reduction potential (ORP), and pH, as non-limiting examples. Since the received raw data is associated with the particular type of sensor 20, 26 used to acquire that data, the cloud-based processing service 22 can be used to analyze the data and generate an output reflective of the sensor output that can be transmitted to the handheld device 28 and/or computer 30 for review by a user.

Each measurement command includes the instructions that define the parameters with which the sensing device 10 collects raw data using the attached wired and/or wireless sensor(s) 20, 26. The measurement command may include information such as a device-specific identification code (i.e., a device ID). For wired sensors 20, the measurement command may include DAS settings that define data acquisition parameters. In some cases, the DAS settings include DFT settings that define the core inputs to the DFT algorithm stored in the DAS 16. The DAS settings associated with the particular measurement command dictate the parameters under which data is acquired using the sensor 20, 26. The DAS settings may dictate an applied voltage potential, current, frequency, duration of time during which data is acquired with the sensor, period of time in which the sample is stimulated before acquiring data, a voltage peak-peak stimulation, internal gain, external gain selections (e.g., a feedback resistor), and/or physical port selection, as non-limiting examples. Where certain types of sensors are used to acquire data, the DAS settings may also include DFT settings associated with the particular measurement command may include a starting stimulation frequency, frequency increments, a number of samples or sample count, as non-limiting examples. It is to be understood that the preceding listing of DAS settings is provided herein as non-limiting examples and that the measurement command may include other DAS settings dictated based on a particular sensor type, application, or desired water quality characteristic being measured. In the case of a virtual or wireless sensor 26, the measurement command may include one or more DAS settings determined by the hardware of the virtual sensor 26. For example, the DAS setting(s) may cause the DAS 16 to receive a continuous stream of data from remote sensor 26 at virtual port 24. As another example, the DAS setting(s) may cause the DAS 16 to ping the remote sensor 26 to acquire/transmit data at certain predefined intervals and/or at a predefined frequency.

The particular DAS settings are dictated based on the type of sensor used and the type of data desired. As one example, if the sensor is a dielectric sensor, the DAS settings would include DFT settings to enable device 10 to acquire raw DFT data and may also include one or more additional DAS settings. On the other hand, in the case of a pH sensor, the DAS settings might include a static current value and no DFT settings.

In some embodiments, technique 300 includes an optional step 308 (shown in phantom) wherein the cloud-based processing service 22 queries remote database 32 for stored information relevant to the attached wired and/or wireless sensor(s) 20, 26. As one example, the cloud-based processing service 22 may query the remote database 32 for calibration values specific to a particular sensor 20, 26. Technique 300 may utilize information stored within the remote database 32 when generating the measurement command and/or when processing the raw data acquired using the particular sensor 20, 26.

At step 310, the cloud-based processing service 22 parses the generated measurement command and determines whether the measurement request is valid. Step 310 may entail verifying one or more items including verifying whether the sensing device 10 is online, verifying whether the sensing device 10 includes appropriate hardware to support the acquisition of data in accordance with the DAS settings defined for a wired sensor 20 or the sensor-specific settings defined for a wireless sensor 26.

After the measurement command is validated, the cloud-based processing service 22 transmits the measurement command to the sensing device 10 at step 312. As described above, cloud-based processing service 22 creates a unique ID or key prior to transmitting the measurement command that tags data received from device 10 following the measurement command to enable cloud-based processing service 22 to determine whether the requested data corresponding to the measurement command was received by the cloud service 22. In one exemplary embodiment, the measurement command is transmitted as an MQTTS command. The transmitted measurement command defines a particular internal hardware configuration of the water sensing device 10 to be used for sensor data acquisition. Sensing device 10 receives the transmitted command and executes the command at step 314 by configuring the internal hardware of the water sensing device 10 to acquire raw data in accordance with the transmitted command.

In the case of a wired sensor, sensing device 10 parses and executes the DAS settings and the DFT settings (if applicable) during step 314. Upon receipt of the DAS settings, sensing device 10 may set up appropriate switching for the external gain feedback resistor, set up AD5934 internal gain, and/or set up AD5934 peak-to-peak voltage stimulation setting, as examples. With respect to the DFT settings, the settings may define a start frequency, define frequency increments, and/or define a number of data samples to acquire, as examples. In the case of a wireless sensor 26, the transmitted command would be used by sensing device 10 to configure data acquisition using the wireless sensor 26 in accordance with the sensor-specific settings determined based on the particular hardware configuration of the wireless sensor 26.

After sensing device 10 is configured to acquire data in accordance with the measurement command, sensing device 10 operates in accordance with the defined configuration to gather raw data at step 316 using the wired and/or wireless sensor(s) 20, 26. This raw data is continuously transmitted by sensing device 10 to cloud-based processing service 22 at step 318 as the data is acquired. The raw data is not stored locally on sensing device 10. In some embodiments, the raw data is transmitted to cloud-based processing service 22 via a MQTTS publish command.

At step 320, the cloud-based processing service 22 transforms the acquired raw sensor data into processed sensor data that can be used to determine water quality characteristics. This data transformation and processing step is continuously or intermittently carried out as additional raw sensor data is transmitted from sensing device 10 to the cloud-based processing service 22, thereby permitting the water characteristics to be monitored over time. In one embodiment, the data processing includes computing actual magnitude and phase of the acquired data. Alternatively, data processing may include converting the acquired measurement (e.g., a point impedance value acquired at a single frequency) using a predefined formula stored on the remote database 32 and/or the cloud-based processing service 22. In yet another embodiment, the data processing may entail comparing acquired data to a look-up table stored on the remote database 32 and/or the cloud-based processing service 22, with the look-up table including values specific to the type or model of sensor used to acquire data. As one example, the look-up table may include pH values corresponding to data measurements acquired from a pH sensor.

According to one embodiment of the invention, the data accumulated by sensing device 10 and processed using cloud-based processing service 22 is continuously output to a database 32 for storage at step 322. Thereafter, the stored data can be accessed by a user through a handheld device 28 or computer 30.

According to one embodiment, the determined water quality characteristics are continuously monitored for outlying data at step 324, which may reflect a particular contaminant or other undesirable characteristic of the monitored water. Since water quality parameters are monitored in real time and over a period of time, average values may be calculated and alerts may be generated at step 326 over the handheld device 28 or computer 30 if the real time value of a particular water quality parameter evidences an abrupt deviation from the tracked average. The GUI 400 (FIG. 2) of handheld device 28 or computer 30 may also include one or more user input sections that permit a user to manually set threshold values for various water quality parameters. In such case, an alert or alarm on the handheld device 28 or computer 30 would be generated when the particular water quality parameter exceeded the predetermined threshold.

In one embodiment where wired sensor 20 is a conductivity sensor coupled to one of the physical ports 18 of sensing device 10, data acquisition system 16 may be controlled in accordance with a measurement command that defines a frequency sweeping protocol or topic during which the conductivity sensor is operated to acquire data across a predetermined frequency range. In one embodiment, this frequency range may span a bandwidth of 300 Hz to 500 kHz. However, it is contemplated that the particular frequency range used to acquire data may be selected based on the particular sensor used and application and, as such, may encompass a band having lower and/or higher frequencies than that previously listed.

The cloud-based processing service 22 accumulates raw physical DFT measurements acquired by the sensing device 10 across the frequency band and over an extended period of time. The resulting DFT measurements represents three or more dimensions of data across the frequency spectrum. The cloud-based processing service 22 converts physical measurements received from the DAS 16 into context-based information that reflects the value of the underlying contaminants detected, a description of the problem, and contextual cues for resolving the problem. The converted/processed data is then stored in the database 32. The converted/processed data corresponding to the acquired raw data from numerous frequency sweeps are used to generate a mass spectrometry model or impedance spectroscopy model, which is stored on the remote network or database 32. Data for these models may be acquired from a single sensing device 10 or a network of sensing devices, all configured similarly to sensing device 10 and located at different geographical areas. Once generated, this regression model may be used to analyze the water composition based on data acquired during a single frequency sweep over the predetermined bandwidth.

As one general example, the generated model might correlate peaks within the conductivity readings acquired at frequency(x) of the sweep as corresponding to the relative concentration of a first contaminant in the water, peaks within the conductivity readings acquired at frequency(y) of the sweep as corresponding to the relative concentration of a second contaminant in the water, and so on. These correlations may then be applied to identify the particular concentration of these different contaminants in a water sample analyzed by the sensing device 10 in real time.

Because raw data is continuously being acquired from sensing device 10 or a network of such sensing devices, the regression model is dynamic in that it changes by learning and improving with increased data. The model is generated in real-time in that the model can be immediately updated with raw data measurements received from multiple sensors coupled to an array of devices. As the model is updated and refined over time, hardware configurations of the sensing device 10 can be updated then by emerging requirements from the cognitive components. For example, responsive to a change in the regressive model, cloud-based processing service 22 may change the hardware configuration of the sensing device 10 in a manner that adjusts operation of a particular sensor in a manner that changes the range of frequencies that a sample is subjected to. These frequencies could range from electric to acoustic to light scatter methods, etc. Rather than a static model of frequency sweeping, where a solution was subjected to a range of x mHz to y mHz in z mHz intervals, the model or algorithm can result in a hardware configuration change that adjusts the frequency range sampled, the intervals sampled, or both.

The mass spectroscopy data acquisition and processing technique described above with respect to conductivity can also be extended to monitor/read other parameters of a medium using alternative sensor components and/or appropriate DAS settings (e.g., gain, voltage, and any of the other inputs listed above). As one example, the technique can be used to measure hardness by transmitting a measurement command via the cloud-based processing service 22 that dictates a wideband frequency sweep (e.g., over a frequency range from 3 kHz to 100 kHz as one non-limiting example). The raw data acquired from sensing device 10 would be processed by the cloud-based service 22 using a transfer function that includes ion exchange, which permits visualizing change in magnitude, takes into account changes in the data across a spectrum of frequencies (i.e., the contour of the mapped data), and also accounts for how much of the total conductivity is represented by the divalent calcium ion. The resulting mass spectroscopy model or transfer function would be location specific to where the initial data was gathered and enable subsequent monitoring and analysis of hardness at the given location.

A similar technique may be used to define a transfer function that enables monitoring/sensing of iron, sulfur/sulfide or any other specific component or contaminant in a monitored medium. The transfer function would be defined by machine learning carried out in the cloud service 22 using raw sensor data acquired during a frequency sweep. Changes in the data acquired during the sweep (e.g., changes in terms of phase or magnitude or some combination thereof) would be analyzed and used to isolate the signatures that are unique to the compound in question. Similar data acquisition and mass spectroscopy modeling techniques may also be utilized to define a transfer function that models any closed system. For example, a transfer function may be developed to model a water softener, a reverse osmosis system, an iron filter, or to monitor depletion/fatigue of self-sacrificing media tanks that are being used to neutralize water. In any of the above examples, the transfer function would be developed over time by identifying “normal” operation of the modeled system via machine learning. The cloud-based service 22 would utilize the modeled values identified as “normal” to analyze later-acquired sensor data. More specifically, the cloud-based service 22 would be configured to compare later-acquired and/or real-time data to the “normal” model and identify abnormalities based on the comparison.

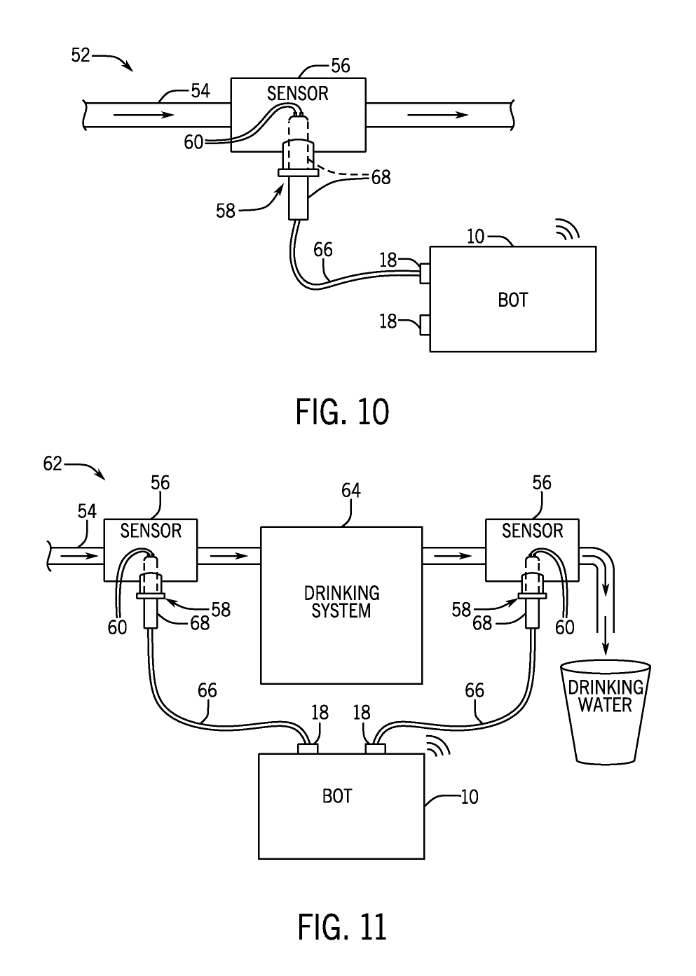

Referring now to FIGS. 10 and 11, alternative implementations utilizing sensing device 10 as a water sensing device are provided. In each implementation, the output of sensing device 10 may be utilized in the manner described above to provide real-time water quality data to a remote user and to alert a user against fluctuations in water quality. FIG. 10 is a schematic illustration of a water sensing system 52 that includes a water sensing device 10 configured to monitor real-time water quality of water flowing through a water supply pipe 54. Water sensing device 10 includes a wired sensor 20 that is coupled to a physical port 18. A tee fitting 56 is integrated onto the water supply pipe 54 to provide an input port 58 into which the wired sensor 20 may inserted. In the case of a conductivity sensor, the wired sensor 20 includes a pair of electrodes 60 that extend into the water stream.

FIG. 11 is a schematic illustration of a water sensing system 62 for use with an existing water purification or drinking system 64, such as, for example, a reverse osmosis system. In system 62, two tee fittings 56 are provided—one on the input side of the drinking system 64 and one on the output side of the drinking system 64. Separate sensors 20 are connected to respective physical ports 18 of water sensing device 10 and are used to monitor the water quality entering and exiting the drinking system 64, respectively. Data acquired from the wired sensor 20 on the inlet side of the drinking system 64 may be used to monitor parameters of the incoming water from the municipality, data acquired from the wired sensor 20 on the outlet side of the drinking system 64 may be used to monitor parameters of the filtered water, and data from both sensors may be compared to assess the relative effectiveness and operation of the drinking system 64. With respect to the later calculation, the effectiveness of the drinking system 64 may be monitored over time. As the effectiveness calculation decreases, the system may be configured to generate an alert that will be sent to the end user and/or a private water treatment service indicating that the drinking system 64 may be in need of service.

An exemplary three-dimensional photographic rendering of sensing device 10 incorporated within water sensing system 62 is provided in FIG. 10, according to one embodiment of the invention. Each wired sensor 20 includes a connection wire 66 having a first end configured to be received within one of the physical ports 18 of sensing device 10. The sensor components (for example electrodes in the case of a conductivity sensor) are provided within a housing 68 positioned at a second end of the wire 66. This housing 68 is constructed to mate couple to and form a watertight seal with the tee-fitting 56.

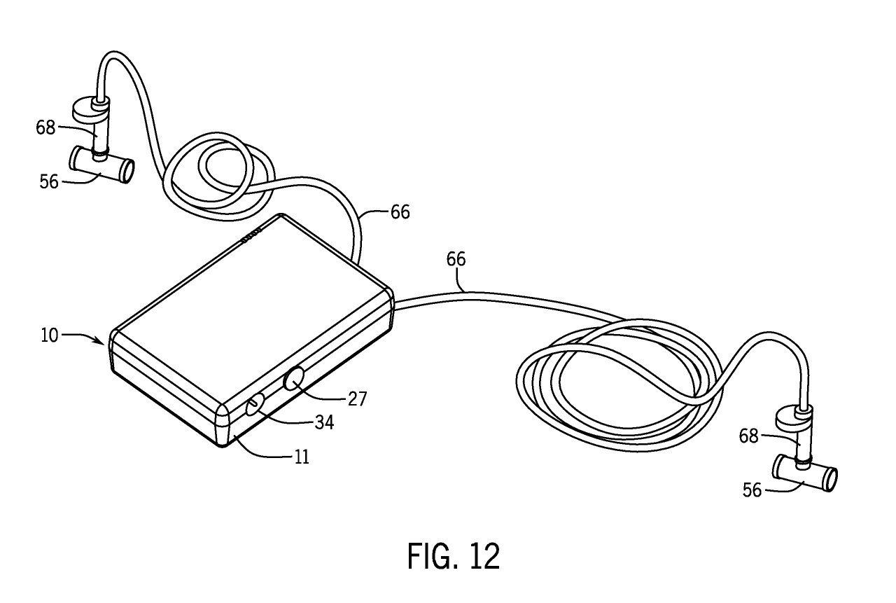

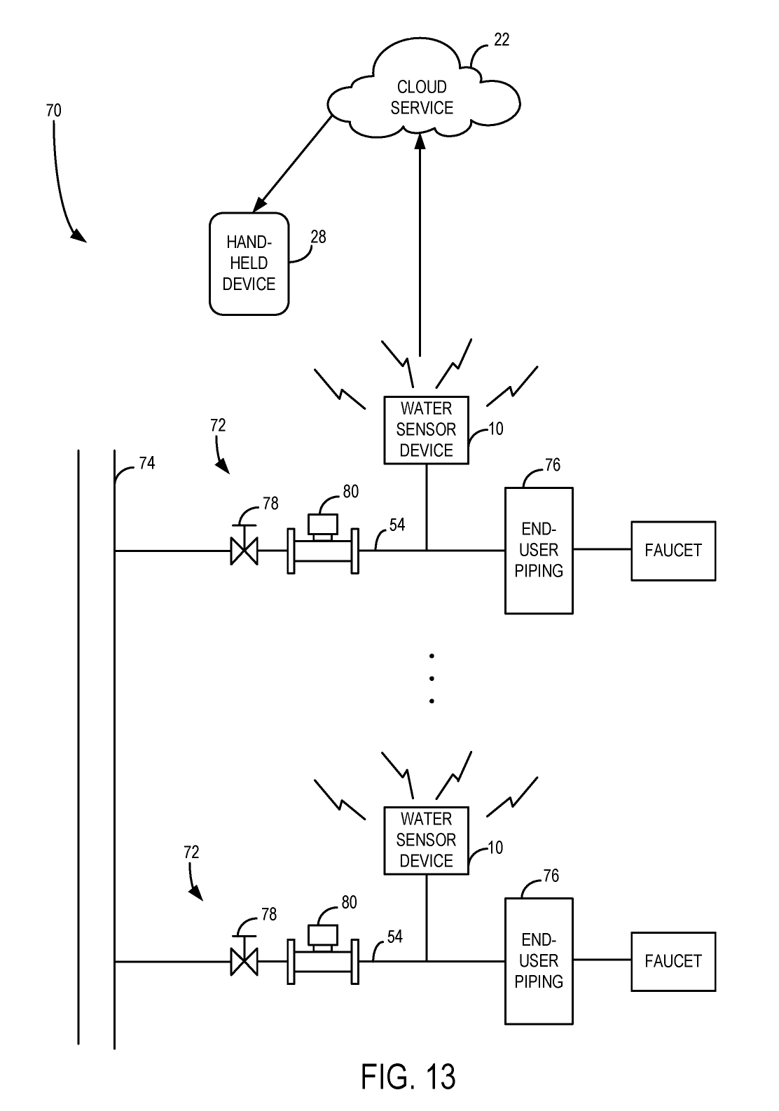

The continuous and real-time data monitoring functionality of the water sensing device 10 described herein may be leveraged within a large-scale system, such as the exemplary water monitoring network 70 generally illustrated in FIG. 12. As shown, water sensing devices 10 are provided at multiple end user locations 72 coupled to a common water distribution main pipe 74, which carries water from a main, municipal water supply. Water from the water distribution main pipe 74 enters the end user's pipe 76 through a shut off valve 78 and a water meter 80. The water sensing device 10 is positioned in-line on the water distribution main pipe 74 between the end user pipe 76 and the water meter 80 and is controlled to acquire raw data corresponding to water quality characteristics that is transmitted to cloud-based processing service 22 and accessed through a handheld device 28 or computer 30 in the manner described above.

The water quality information generated by cloud-based processing service 22 may be accessed by one of the particular end users receiving water from the water distribution main pipe 74. Alternatively, the acquired and processed data may be accessed by a municipality or private water treatment service to monitor water quality throughout the network 70. By continuously monitoring changes in the water supply, the municipality or water treatment service can identify and treat problems within the water supply before the problems expand in scale and severity. Also, by comparing the relative water quality readings corresponding to the different water sensing devices 10 within the network 70, the municipality or service provider is able to pinpoint the likely location that the contaminant is entering the water supply. For example, elevated levels of a particular contaminant in the water supplies of a series of end users located near a construction site might be used to identify the location of a break in the water line resulting from the construction activities. This information can significantly reduce the costs and timeframe for remediation.

The sensor data output from a particular end-user's water sensing device 10 may also be accessed and monitored by the end-user's private water treatment service. Based on this monitored data, the treatment service may schedule a service call to diagnose the cause of elevated levels of contaminants, deliver additional salt if the load sensor on the user's brine tank indicates that the current salt supply is low, or perform any number of other water treatment services.

In addition to monitoring residential drinking water quality, it is contemplated that sensing device 10 may be used for other applications, including for example remote monitoring of swimming pool and spa water quality, operational characteristics of a water softener, and depletion/fatigue of self-sacrificing media tanks that are being used to neutralize water.

In the embodiments described above, the sensing device 10 is controlled to acquire data from an attached sensor 20, 26 according to data acquisition parameters that are received from the cloud-based processing service 22. Thus, the defined hardware configuration for acquiring data with a particular sensor is stored on the cloud-based service 22 and not reside on the device 10 itself. When the particular sensor 20, 26 is coupled to the device 10, the cloud-based processing service 22 determines appropriate DAS settings for that sensor 20, 26 and transmits those settings to the device 10. The DAS settings dictate how the internal hardware components of device 10 operate to acquire raw data using the attached sensor 20, 26. Since device 10 does not include any stored software for acquiring measurements from a particular sensor 20, 26, device 10 cannot acquire meaningful data with that particular sensor 20, 26 absent receipt of a measurement command from the cloud-based service 22.

In an alternative embodiment, the microprocessor 121 on device 10 may include stored programming that defines a predefined and static hardware configuration and accompanying DAS settings that enables device 10 to acquire data corresponding to a particular water quality parameter such as, for example, a conductivity measurement. Additionally, in alternative embodiments sensing device 10 may include additional data storage either within an alternative configuration of the microcontroller itself or another external component. For example, sensing device 10 may include an optional SD card either coupled directly to the circuit board itself or coupleable to the device 10 through a slot in the housing 11. Additional data storage capabilities on the sensing device 10 itself may be used to enable some storage of acquired raw sensor data and/or the ability to download executable instructions to the device 10. In the latter case, the downloaded executable instructions may include control software or scripts that include measurement commands similar to that described above with respect to the cloud-based processing service 22. In other words, the downloaded executable instructions would be used by the device 10 itself to generate the measurement command, which would then be executed on the device 10 by the microprocessor 121 configuring the device hardware according to the DAS setting(s) associated with the measurement command. The actual initiation of a data measurement may occur on the device 10 rather than based on an instruction received from the cloud service 22. To accomplish this, the device 10 may be programmed with software that includes a timer or loop, which executes the instructions corresponding to a measurement command that is stored in a local file on the device 10. Data acquisition and processing would otherwise occur in a similar fashion as described with respect to technique 300 of FIG. 9.

Beneficially, in some of the embodiments of the invention disclosed herein provide a sensing device 10 that is agnostic to the type of sensor attached to the device. In essence, device 10 is a “dumb” device in the sense that it does not include any core programming that enables the device 10 to acquire meaningful data when a wired or wireless sensor 20, 26 is attached to the device 10. The hardware controls that dictate how the device 10 acquires data using a particular sensor 20, 26 reside in the cloud-based processing service 22. This means that device 10 can be controlled to acquire data using virtually any type of sensor. As new sensors are developed, measurement commands for those new sensors can be added to the cloud-based processing service 22 and/or remote database 32 thereby enabling these new sensors to be used in connection with device 10.

One skilled in the art will appreciate that embodiments of the invention may be interfaced to and controlled by a computer readable storage medium having stored thereon a computer program. The computer readable storage medium includes a plurality of components such as one or more of electronic components, hardware components, and/or computer software components. These components may include one or more computer readable storage media that generally stores instructions such as software, firmware and/or assembly language for performing one or more portions of one or more implementations or embodiments of a sequence. These computer readable storage media are generally non-transitory and/or tangible. Examples of such a computer readable storage medium include a recordable data storage medium of a computer and/or storage device. The computer readable storage media may employ, for example, one or more of a magnetic, electrical, optical, biological, and/or atomic data storage medium. Further, such media may take the form of, for example, floppy disks, magnetic tapes, CD-ROMs, DVD-ROMs, hard disk drives, and/or electronic memory. Other forms of non-transitory and/or tangible computer readable storage media not list may be employed with embodiments of the invention.

According to one embodiment, a non-transitory computer readable storage medium has stored thereon a computer program comprising instructions which, when executed by a controller (e.g., microcontroller 121), cause the controller to interpret a measurement command received from an external source (e.g., cloud-based processing service 22) as defining at least one data acquisition system (DAS) setting configured to stimulate a sensor. The instructions further cause the controller to configure at least one DAS component of a sensing device coupled to the sensor in accordance with a first DAS setting of the at least one DAS setting, receive data from the stimulated sensor, and transmit the received data to the external source. The instructions may also cause the controller to locate a topic identifier within the measurement command, associate the topic identifier with a setting protocol stored on the controller, and interpret the first DAS setting based on the setting protocol.

A number of such components can be combined or divided in an implementation of a system. Further, such components may include a set and/or series of computer instructions written in or implemented with any of a number of programming languages, as will be appreciated by those skilled in the art. In addition, other forms of computer readable media such as a carrier wave may be employed to embody a computer data signal representing a sequence of instructions that when executed by one or more computers causes the one or more computers to perform one or more portions of one or more implementations or embodiments of a sequence.

Therefore, according to one embodiment of the invention, a sensing device includes at least one input port, a data acquisition system (DAS) in communication with the at least one input port, and a main connectivity module coupled to the DAS. The main connectivity module is configured to receive a measurement command from a source external to the sensing device. The measurement command includes at least one DAS setting specific to a type of sensor coupled to the at least one input port. The main connectivity module is also configured to execute the at least one DAS setting by causing the DAS to stimulate a sensor coupled to the at least one input port, receive raw data from the stimulated sensor, and transmit the raw data to a processor external to the sensing device.

According to another embodiment of the invention, a remotely-controlled sensor system includes a remote database and a sensing device having a main connectivity module, a data acquisition system (DAS) in communication with the main connectivity module, and a first input port in communication with the DAS. The first sensor is coupled to the input port. The system also includes a data processing system in communication with the sensing device and the remote database. The data processing system includes a processor configured or programmed to identify a sensor type of the sensor using data stored on the remote database and generate a measurement command based on the identified sensor type, where the measurement command includes at least one DAS setting that defines a hardware configuration for the sensing device. The processor is also configured or programmed to transmit the measurement command to the sensing device and receive raw data from the sensing device, where the raw data includes data acquired by the stimulating the sensor in accordance with the at least one DAS setting.

According to yet another embodiment of the invention, a method of acquiring data from a sensor includes identifying a sensor type of a sensor coupled to a sensing device using data stored within a database and generating a measurement command within a processor external to the sensing device, the measurement command including at least one data acquisition system (DAS) setting configured to stimulate the sensor based on the identified sensor type. The method also includes transmitting the measurement command from the external processor to a microcontroller on the sensing device and executing the measurement command within the microcontroller by configuring at least one DAS component on the sensing device to stimulate the sensor in accordance with the at least one DAS setting. The method further includes receiving raw data from the stimulated sensor at the sensing device and transmitting the raw data from the sensing device to the external processor in real time.

While the invention has been described in detail in connection with only a limited number of embodiments, it should be readily understood that the invention is not limited to such disclosed embodiments. Rather, the invention can be modified to incorporate any number of variations, alterations, substitutions or equivalent arrangements not heretofore described, but which are commensurate with the spirit and scope of the invention. Additionally, while various embodiments of the invention have been described, it is to be understood that aspects of the invention may include only some of the described embodiments. Accordingly, the invention is not to be seen as limited by the foregoing description, but is only limited by the scope of the appended claims.

What is claimed is:

1. A sensing device comprising:
at least one input port;
a data acquisition system (DAS) in communication with the at least one input port; and
a main connectivity module coupled to the DAS, the main connectivity module configured to:
receive a measurement command from a source external to the sensing device, the measurement command comprising at least one DAS setting specific to a type of sensor coupled to the at least one input port;
execute the at least one DAS setting by causing the DAS to stimulate a sensor coupled to the at least one input port;
receive raw data from the stimulated sensor; and
transmit the raw data to a processor external to the sensing device.

2. The sensing device of claim 1 wherein the at least one DAS setting further comprises at least one Discrete Fourier Transform (DFT) setting; and
wherein the DAS is configured to:
apply an DFT algorithm on the raw data according to the at least one DFT setting; and
transmit raw DFT data to the main connectivity module.

3. The sensing device of claim 1 wherein the at least one input port comprises at least a first input port and a second input port.

4. The sensing device of claim 3 wherein the main connectivity module is configured to:
receive a first measurement command from the external source, the first measurement command comprising a first set of DAS settings configured to stimulate a first sensor coupled to first input port;
execute the first set of DAS settings by causing the DAS to stimulate the first sensor;
execute the second set of DAS settings by causing the DAS to stimulate the second sensor;
receive raw data from the first sensor and the second sensor; and
transmit the raw data from the first sensor and the second sensor to the external processor.

5. The sensing device of claim 1 further comprising a multiplexing system; and
wherein the at least one DAS setting defines a hardware configuration of the DAS and the multiplexing system that enables the sensing device to acquire data from an attached sensor.

6. The sensing device of claim 1 wherein the at least one DAS setting comprises at least one of a frequency value, a voltage value, a sample count, and a frequency band over which to stimulate the sensor.

7. The sensing device of claim 1 wherein the main connectivity module comprises a microcontroller; and
wherein the microcontroller comprises a hardware API that executes the at least one DAS setting in real time within the sensing device.

8. The sensing device of claim 1 wherein the sensing device comprises a sensor agnostic device.

9. The sensing device of claim 1 wherein the main connectivity module is configured to transmit the raw data to the external source in real time via a persistent wireless connection with the external source.

10. A remotely-controlled sensor system comprising:
a remote database;
a sensing device comprising:
a main connectivity module;
a data acquisition system (DAS) in communication with the main connectivity module; and
a first input port in communication with the DAS;
a first sensor coupled to the input port;
a data processing system in communication with the sensing device and the remote database, the data processing system comprising a processor configured to:
identify a sensor type of the sensor using data stored on the remote database;
generate a measurement command based on the identified sensor type, the measurement command comprising at least one DAS setting that defines a hardware configuration for the sensing device;
transmit the measurement command to the sensing device; and
receive raw data from the sensing device, the raw data comprising data acquired by the stimulating the sensor in accordance with the at least one DAS setting.

11. The remotely-controlled sensor system of claim 10 wherein the measurement command comprises a topic identifier and the at least one DAS setting; and
wherein the sensing device comprises a hardware API that executes the at least one DAS setting based on the topic identifier.

12. The remotely-controlled sensor system of claim 11 wherein the at least one DAS setting comprises at least one DFT setting.

13. The remotely-controlled sensor system of claim 10 wherein the sensing device further comprises a multiplexing system; and
wherein the at least one DAS setting comprises a plurality of DAS settings that define a data acquisition configuration for the DAS and the multiplexing system.

14. The remotely-controlled sensor system of claim 10 wherein the data processing system comprises programming that defines a plurality of measurement commands.

15. The remotely-controlled sensor system of claim 10 wherein the measurement command configures the sensing device to sweep a frequency spectrum.

16. The remotely-controlled sensor system of claim 10 wherein the data processing system comprises a calibration script that defines an initial calibration procedure for the sensing device; and
wherein the calibration script populates an initial database for the sensing device and transmits a measurement command that initiates a series of calibration readings from one or more components on the sensing device.

17. The remotely-controlled sensor system of claim 10 further comprising a graphical user interface (GUI) that includes an input field that permits a user to input the sensor type of the sensor; and
wherein the data processing system identifies the sensor type of the sensor based on the user input.

18. A method of acquiring data from a sensor comprising:
identifying a sensor type of a sensor coupled to a sensing device using data stored within a database;
generating a measurement command within a processor external to the sensing device, the measurement command comprising at least one data acquisition system (DAS) setting configured to stimulate the sensor based on the identified sensor type;
transmitting the measurement command from the external processor to a microcontroller on the sensing device;
executing the measurement command within the microcontroller by configuring at least one DAS component on the sensing device to stimulate the sensor in accordance with the at least one DAS setting;
receiving raw data from the stimulated sensor at the sensing device; and
transmitting the raw data from the sensing device to the external processor in real time.

19. The method of claim 18 further comprising generating the measurement command to include at least one Discrete Fourier Transform (DFT) setting that defines an input to an DFT algorithm stored in a DAS of the sensing device.

20. The method of claim 18 further comprising generating the measurement command to include a DAS setting that configures the at least one DAS component on the sensing device to stimulate the sensor over a band of frequencies.
